(19) United States
(12) Patent Application Publication
(10) Pub. No.: US 2007/0026026 A1
(43) Pub. Date: Feb. 1, 2007

(54) Oral liquid losartan compositions

(76) Inventors: David Delmarre, Vernon Hills, IL (US); Naga Mallika Surapaneni, Mundelein, IL (US); Carlos-Julian Sison Centeno, Chicago, IL (US); Meagan Erica Weldele, Overland Parks, KS (US); Danchen Gao, Chicago, IL (US)

Correspondence Address: WINSTON & STRAWN LLP;PATENT DEPARTMENT, 1700 K STREET, N.W., WASHINGTON, DC, 20006, US

(21) Appl. No.: 11/493,801

(22) Filed: Jul. 27, 2006

Related U.S. Application Data
(60) Provisional application No. 60/703,866, filed on Aug. 1, 2005.

(51) Int. Cl. A61K 31/4184; A61K 9/00

(52) U.S. Cl. 424/400; 514/381

(57) ABSTRACT

The present invention is directed to improved oral liquid compositions that include losartan, or a pharmaceutically acceptable salt or metabolite thereof, and at least one pharmaceutically acceptable carrier in an amount sufficient to provide a pH of about 6 or higher. Processes of preparing such compositions and methods of administering such compositions are also included.

### CROSS-REFERENCE TO RELATED APPLICATIONS

This application claims the benefit of provisional application No. 60/703,866, filed Aug. 1, 2005, the entire contents of which is incorporated herein by express reference thereto.

### TECHNICAL FIELD

The present invention relates generally to oral liquid losartan compositions and processes for their preparation. The invention further relates to methods of treatment by administering the compositions.

### BACKGROUND OF THE INVENTION

Losartan potassium, a pharmaceutically acceptable salt of losartan, is a white to off-white free flowing crystalline powder with a molecular weight of 461.01. It is freely soluble in water, soluble in alcohols, slightly soluble in common organic solvents, such as acetonitrile and methylethyl ketone, and practically insoluble in chloroform. The melting point of losartan potassium is between 270° C. to 276° C. Oxidation of the 5-hydroxymethyl group on the imidazole ring results in the active metabolite of losartan.

Losartan potassium, the first of a new class of antihypertensives, is an angiotensin II receptor (type AT₁) antagonist. Angiotensin II, which is formed from angiotensin I in a reaction catalyzed by angiotensin converting enzyme (ACE, kinase II), is a potent vasoconstrictor, the primary vasoactive hormone of the renin-angiotensin system and an important component in the pathophysiology of hypertension. It also stimulates aldosterone secretion by the adrenal cortex.

Losartan potassium is also known as 2-butyl-4-chloro-1-[p-(o-1H-tetrazol-5-ylphenyl)benzyl]imidazole-5-methanol monopotassium salt. Its empirical formula is C₂₂H₂₂ClKN₆O and it has the following structural formula:

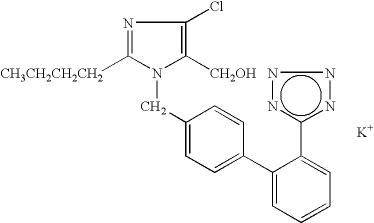

Losartan is an active agent provided in solid oral dose forms that undergoes substantial first-pass metabolism by cytochrome P450 enzymes. It is converted, in part, to an active carboxylic acid metabolite that is responsible for most of the angiotensin II receptor antagonism that follows losartan treatment. The terminal half-life of losartan is about 2 hours and of the metabolite is about 6 to 9 hours. The pharmacokinetics of losartan and its active metabolite are linear with oral losartan doses up to 200 mg and do not change over time.

Losartan and its principal active metabolite block the vasoconstrictor and aldosterone-secreting effects of angiotensin II by selectively blocking the binding of angiotensin II to the AT₁ receptor found in many tissues, (e.g., vascular smooth muscle, adrenal gland). There is also an AT₂ receptor found in many tissues, but it is not known to be associated with cardiovascular homeostasis. Both losartan and its principal active metabolite do not exhibit any partial agonist activity at the AT₁ receptor and have much greater affinity (about 1000-fold) for the AT₁ receptor than for the AT₂ receptor. In vitro binding studies indicate that losartan is a reversible, competitive inhibitor of the AT₁ receptor. The active metabolite is 10 to 40 times more potent by weight than losartan and appears to be a reversible, non-competitive inhibitor of the AT₁ receptor.

Following oral administration, the systemic bioavailability of losartan is approximately 33 percent. About 14 percent of an orally-administered dose of losartan is converted to the active metabolite. Mean peak concentrations of losartan and its active metabolite are reached in 1 hour and in 3 to 4 hours, respectively. While maximum plasma concentrations of losartan and its active metabolite are approximately equal, the AUC of the metabolite is about 4 times as great as that of losartan. A meal slows absorption of losartan and decreases its Cₘₐₓ, but has only minor effects on losartan AUC or on the AUC of the metabolite (about 10 percent decreased).

Losartan metabolites have been identified in human plasma and urine. In addition to the active carboxylic acid metabolite, several inactive metabolites are formed. Following solid oral and intravenous administration of ¹⁴C-labeled losartan potassium, circulating plasma radioactivity is primarily attributed to losartan and its active metabolite. In vitro studies indicate that cytochrome P450 2C9 and 3A4 are involved in the biotransformation of losartan to its metabolites. Minimal conversion of losartan to the active metabolite (less than 1 percent of the dose compared to 14 percent of the dose in normal subjects) was seen in about 1 percent of individuals studied.

Losartan potassium is indicated for the treatment of hypertension. It may be used alone or in combination with other antihypertensive agents. The usual starting dose of losartan potassium is 50 mg once daily, with 25 mg used in patients with possible depletion of intravascular volume (e.g., patients treated with diuretics) and patients with a history of hepatic impairment. Losartan potassium can be administered once or twice daily with total daily doses ranging from 25 mg to 100 mg.

If the antihypertensive effect measured at trough using once-a-day dosing is inadequate, a twice-a-day regimen at the same total daily dose or an increase in dose may give a more satisfactory response. If blood pressure is not controlled by losartan potassium alone, a low dose of a diuretic may be added.

U.S. Pat. No. 5,138,069 describes and claims a generic active compound that encompasses losartan. The '069 patent describes the active compound as a useful angiotensin II blocker, and its activity in treating hypertension and congestive heart failure. The '069 patent alleges that active ingredient formulations can be administered through any typical route of administration including liquid dosage forms for oral administration, and that dosage forms can contain coloring and flavoring to increase patience acceptance.

The '069 patent, related U.S. Pat. Nos. 5,153,197 and 5,210,079, and unrelated U.S. Pat. No. 5,608,075, each specifically describe the formulation of the active ingredient into capsules, tablets, injectable solutions, and suspensions. An aqueous suspension of the active ingredient is prepared so that each 5 mL contains 100 mg of the active ingredient, 100 mg of sodium carboxymethylcellulose, 5 mg of sodium benzoate, 1.0 g of sorbitol solution, U.S.P., and 0.025 mL of vanillin.

The '197 patent noted above claims methods of treating hypertension by administering losartan and pharmaceutical compositions comprising a pharmaceutically acceptable carrier, a diuretic and losartan. The '079 patent noted above discloses the use of losartan for treating chronic renal failure. The '075 patent noted above discloses distinct crystalline structures, or forms, of losartan potassium which were designated Form I and Form II based on their respective thermal stability. Form I is a low-temperature stable form, and Form II is a high temperature stable form. Form I has been shown to be the more thermodynamically stable polymorph at room temperature.

U.S. Pat. No. 5,266,583 discloses and claims a metabolite of losartan. The metabolite may be utilized in compositions such as tablets, capsules, or elixirs for oral administration. A syrup or elixir may contain the metabolite, sucrose as a sweetening agent, methyl and propyl parabens as preservatives, a dye, and a flavoring such as cherry or orange flavor.

In 1995, losartan became the first nonpeptide AT₁ antagonist approved by the U.S. Food and Drug Administration for clinical use. In particular, losartan is approved for the treatment of hypertension alone or in combination with other antihypertensive agents. Losartan may be administered orally as its mono-potassium salt. Losartan potassium is available by prescription in tablet form as a sole active ingredient (Cozaar™, Merck) and as a co-active ingredient with hydrochlorothiazide (Hyzaar™, Merck). Cozaar™ is available as tablets for oral administration containing either 25 mg, 50 mg or 100 mg of losartan potassium and the following inactive ingredients: microcrystalline cellulose, lactose hydrous, pregelatinized starch, magnesium stearate, hydroxypropyl cellulose, hydroxypropyl methylcellulose, titanium dioxide, D&C yellow No. 10 aluminum lake and FD&C blue No. 2 aluminum lake.

International Publication No. WO 03/035039 describes methods of treatment for hypertension and other disease states by delivering losartan in a gastric retained dosage form. The dosage form is preferably a tablet or capsule. The '039 publication, however, also describes liquid preparations of losartan. The liquid preparations may be prepared in the form of syrups or suspensions and contain about 0.2-20 weight percent of the active agent, with the remainder consisting of sugar or sugar alcohols and a mixture of ethanol, water, glycerol, propylene glycol and polyethylene glycol. If desired, such liquid preparations may contain colorant agents, flavoring agents, saccharin and carboxymethyl cellulose or other thickening agents. Liquid preparations for oral administration may also be prepared in the form of a dry powder to be reconstituted with a suitable solvent prior to use.

International Publication No. WO 03/048135 relates to amorphous losartan potassium, losartan potassium in a crystalline form that is a hydrate, crystalline losartan potassium Form IV and solvates thereof, and crystalline losartan potassium Form V and solvates thereof. Liquid pharmaceutical compositions of losartan can include losartan dissolved or suspended in a liquid carrier such as water, vegetable oil, alcohol, polyethylene glycol, propylene glycol or glycerin. The compositions can also contain emulsifying agents, viscosity-enhancing agents, sweetening agents, preservatives and chelating agents, and buffers. Capsules or tablets are the preferred oral dosage form.

Because of the ease of preparing solid dosage forms, tablets and capsules are often the preferred dosage form for many drugs including losartan. Indeed, the only FDA approved losartan products are in solid form, i.e., as tablets. Liquid dosage forms present more of a challenge because of the solubility and stability characteristics of the active compound, as well as the various excipients, in different solvents. In view of the foregoing, it would be desirable to have suitable oral liquid losartan compositions with improved dissolution and stability properties as an additional treatment option.

### SUMMARY OF THE INVENTION

The present invention relates to an oral liquid composition that includes losartan, or a pharmaceutically acceptable salt or metabolite thereof, and at least one pharmaceutically acceptable carrier in an amount sufficient to provide a pH of about 6 or higher. In one preferred embodiment, the pH is about 7 to 10. In a more preferred embodiment, the pH is about 7.4 to 8.6.

Typically, the at least one pharmaceutically acceptable carrier includes one or more of a wetting agent, surfactant component, stabilizing agent, solubilizing agent, thickening agent, sweetening agent, flavoring agent, perfuming agent, colorant agent, preservative agent, or buffering agent. Preferably, the carrier includes one or more of a solubilizing agent, a stabilizing agent, a sweetening agent, a flavoring agent, or a buffering agent.

The amounts of the components in the composition can vary. In one embodiment, the solubilizing agent is present in an amount of about 1 percent to 50 percent (v/v). Preferably, the sweetening agent is present in an amount of about 0.05 percent to 5 percent (w/v) or the buffering agent is present in an amount of about 30 percent to 70 percent (v/v).

More preferably, the carrier includes a glycol or glycerin, the sweetening agent includes a sucralose or saccharin-containing component, and the buffering agent includes a phosphate-containing buffer. In another preferred embodiment, the carrier further includes a flavoring agent, which preferably includes a fruit flavor. The fruit flavor may preferably include grapefruit, orange, lemon, lime, mango, strawberry, pineapple, cherry, or a combination thereof. Generally, the glycol is present in an amount of about 1 percent to 40 percent (v/v), and the glycerin is present in an amount of about 5 percent to 50 percent (v/v). In one embodiment, the phosphate-containing buffer includes sodium phosphate, potassium phosphate, or mixtures thereof. The concentration of phosphate salt in the buffering agent is usually from about 5 mM to 150 mM. In a preferred embodiment, the phosphate salt concentration is about 80 mM to 120 mM. The losartan, or salt or metabolite thereof, is typically present in an amount of about 1 to 50 mg/mL of the composition.

In a preferred embodiment, the losartan, or salt or metabolite thereof, is present in an amount of about 8 mg/mL to 20 mg/mL of the composition, and the pharmaceutically acceptable carrier includes a glycol present in an amount of about 5 percent to 25 percent (v/v), glycerin present in an amount of about 20 percent to 40 percent (v/v), a sweetening agent present in an amount of about 0.5 percent to 2 percent (w/v), and a buffering agent present in an amount of about 40 percent to 60 percent (v/v).

Advantageously, the composition is at least substantially stable. In one embodiment, the degradation of losartan over a period of less than two months is no more than about 1 percent to 2 percent (w/w) at 50° C. In a preferred embodiment, the liquid compositions are substantially stable.

In an exemplary embodiment, the composition is in solution form.

The present invention further relates to an oral liquid composition that includes losartan, or a pharmaceutically acceptable salt or metabolite thereof, at least one of propylene glycol or glycerin, along with a sucralose or saccharin-containing component and a phosphate-containing buffer that includes sodium phosphate, potassium phosphate, or a mixture thereof, in an amount sufficient to provide a pH of about 6 or higher.

The present invention also encompasses stable oral liquid losartan compositions that include losartan or a pharmaceutically acceptable salt or metabolite thereof, a solubilizing agent, a sweetening agent, and a buffering agent, present in an amount sufficient to provide a pH of about 6 or higher over an extended period of time to minimize degradation of the losartan component.

The present invention additionally includes oral liquid compositions that include solutions of losartan, or a pharmaceutically acceptable salt or metabolite thereof, propylene glycol, glycerin, saccharin sodium, and potassium phosphate buffer, each in an amount sufficient to provide a pH of about 6 or higher.

The present invention also relates to a method of preparing an oral liquid losartan composition. The method includes dissolving an amount of losartan, or a pharmaceutically acceptable salt or metabolite thereof, into at least one liquid adjuvant to form a liquid losartan solution, and combining the liquid losartan solution with one or more additional pharmaceutically acceptable carriers sufficient to provide a pH of about 6 or higher to form the oral liquid losartan composition. Preferably, the at least one liquid adjuvant includes a buffering agent. The oral liquid losartan composition is typically a clear solution.

Furthermore, the present invention relates to a method of preventing, treating, or managing hypertension, congestive heart failure, diabetic nephropathy or myocardial infarction in a mammal. The method includes administering to the mammal an effective amount of an oral liquid losartan composition that includes losartan, or a pharmaceutically acceptable salt or metabolite thereof, and at least one pharmaceutically acceptable carrier, wherein the oral liquid losartan composition has a pH of about 6 or higher.

In one embodiment, the composition is administered once or twice a day. In another embodiment, the total daily dose of losartan is from about 25 mg to 100 mg.

The method preferably also includes administering at least one other therapeutic agent in association with the losartan. The other therapeutic agent(s) typically include another hypertensive agent. The at least one other hypertensive agent generally includes an angiotensin II antagonist, angiotensin converting enzyme inhibitor, or a neutral endopeptidase/angiotensin converting enzyme inhibitor, or a combination thereof.

### BRIEF DESCRIPTION OF THE DRAWINGS

Further features and advantages of the invention can be ascertained from the detailed description that is provided below in connection with the following drawing(s):

FIG. 1 is a graph showing the solubility profile of losartan as a function of pH;

FIG. 2 is a graph showing the stability (measured by percent active) of losartan with different buffers at different times;

FIG. 3 is a graph showing the effect of different concentrations of propylene glycol on the stability of losartan;

FIG. 4 is a graph showing the stability of losartan (measured by percent active) with different excipient combinations as a function of time;

FIG. 5 is a graph showing the stability of losartan (measured by percent active) in solutions of propylene glycol, glycerin, and xylitol;

FIG. 6 is a graph showing the stability of losartan (measured by percent active) with different concentrations of glycerin and at different pHs; and

FIG. 7 is a graph showing the percent recovery of losartan at different temperatures in different containers.

### DETAILED DESCRIPTION OF THE PREFERRED EMBODIMENTS

The present invention provides oral liquid compositions that include losartan, or a pharmaceutically acceptable salt or metabolite thereof, and at least one pharmaceutically acceptable carrier in an amount sufficient to provide a pH of about 6 or higher. As used herein, “oral liquid composition(s)” include emulsions, solutions, suspensions, syrups and elixirs, but do not include solid dosage forms that include minor amounts of liquids therein, such as capsules or tablets. Preferably, “oral liquid composition(s)” mean losartan, or a pharmaceutically acceptable salt or metabolite thereof, in solution.

The present liquid dosage forms provide certain advantages over the solid forms conventionally available. For example, liquid dosage forms are much easier to swallow and typically do not require separate water to administer. Patient compliance is also easier to facilitate with the flavoring and colorant agents that are typically included in liquid dosage forms. This is often an issue with very young patients and the elderly. Because the elderly are more likely to suffer from hypertension or high blood pressure than other members of the population, it would be particularly beneficial to have an antihypertensive drug in a palatable oral composition that can help increase patient compliance.

The active ingredient in the present invention is losartan. Losartan is preferably used in the form of a pharmaceutically acceptable salt or metabolite that retains the biological effectiveness and properties of losartan and is not biologically or otherwise undesirable. As used herein, “losartan” includes the agent itself, as well as its pharmaceutically acceptable salts or an active metabolite.

The term “pharmaceutically acceptable salt(s)” or “a pharmaceutically acceptable salt thereof” refers to salt(s) prepared from pharmaceutically acceptable non-toxic acid or bases including inorganic acids and bases and organic acids or bases. The pharmaceutically acceptable salts used in the present invention may be amphoteric, may be present in the form of internal salts, or both.

Losartan may form acid addition salts and salts with bases. Exemplary acids that can be used to form such salts include one or more mineral acids such as hydrochloric, hydrobromic, sulfuric or phosphoric acid or organic acids such as organic sulfonic acids and organic carboxylic acids. Salts formed with inorganic bases include, for example, the sodium, potassium, lithium, ammonium, calcium, and magnesium salts, or any combination thereof. Salts derived from organic bases include, for example, one or more salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethyl aminoethanol, trimethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, fumarate, maleate, succinate, acetate and oxalate.

A particularly suitable pharmaceutically acceptable salt is losartan potassium (C₂₂H₂₂ClKN₆O), which is chemically described as 2-butyl-4-chloro-1[p-(o-1H-tetrazol-5-yl-phenyl)benzyl]imidazole-5-methanol monopotassium salt.

Losartan potassium can be prepared by any suitable method available to one of ordinary skill in the art. For instance, in Example 316, Part D of U.S. Pat. Nos. 5,128,355, 5,138,069 and 5,155,118, each of which are incorporated herein by express reference thereto, losartan is isolated as its potassium salt by crystallization from a mixture of isopropyl alcohol and heptane. The crystals were reported to have a melting point above 250° C.

Another suitable method of preparing a losartan salt is described in Example 5 of U.S. Pat. No. 5,962,500, and in Example 21 of U.S. Pat. Nos. 5,206,374 and 5,310,928, each of which are incorporated herein by express reference thereto. Losartan potassium salt was generated from a solution of losartan by extracting losartan from the solution with an adsorbent, treating the adsorbent with monobasic potassium phosphate and eluting losartan potassium from the adsorbent with 20 percent aqueous THF. The eluent was then concentrated and diluted with isopropyl alcohol, which yielded crystalline losartan potassium. The losartan potassium product was also obtained by spray drying.

Yet another suitable method of preparing a losartan salt is described in Example 8 of U.S. Pat. Nos. 5,130,439, 5,206,374 and 5,310,928, each of which are incorporated herein by express reference thereto. Losartan potassium salt was crystallized from a mixture of isopropyl alcohol, water and heptane. The product was collected by filtration, rinsed with heptane and dried at 50° C. in a vacuum oven to yield a white solid that decomposed at 267° C. to 269° C.

It has now been discovered that the pH of an oral liquid losartan composition should be typically about 6 or higher to provide the surprisingly and unexpectedly improved solubility and stability of losartan in solution achieved by the present invention. In one embodiment, the pH is preferably at least about 7. Preferably, the pH is about −7 to 10 or 7.4 to 10, more preferably about 7.8 to 8.6. An exemplary pH is about 8.2. The pH of the oral liquid composition is preferably adjusted by at least one buffering agent.

The compositions are formed with a pharmaceutically acceptable carrier that preferably includes a pH modifier, such as a buffering agent, to facilitate obtaining a pH of about 6 or more. The compositions of the invention preferably further include one or more wetting agents or surfactants, stabilizing agents, thickening agents, sweetening agents, flavoring agents, perfuming agents, colorant agents, preservative agent, solubilizing agents, isotonizing agents, antioxidant components, and chelating agents. In the case of each of these pharmaceutically acceptable carriers, any suitable type or amount of carrier available to those of ordinary skill in the art may be included according to the invention so long as it does not preclude the losartan from remaining in solution. Therefore, one or more of these carriers can also act as a solvent for losartan. Losartan may be dissolved or suspended in a solvent such as water, vegetable oil, alcohol, polyethylene glycol, propylene glycol or glycerin. Suitable vegetable oils include, e.g., sesame oil, corn oil, olive oil, cottonseed oil, or a mixture thereof.

A wetting agent or surfactant component that can be included in the liquid compositions of the present invention, when used, include one or more quaternary ammonium compounds, such as benzalkonium chloride, benzethonium chloride and cetylpyridinium chloride; TPGS, dioctyl sodium sulfosuccinate; polyoxyethylene alkylphenyl ethers, such as nonoxynol 9, nonoxynol 10, and octoxynol 9; poloxamers (polyoxyethylene and polyoxypropylene block copolymers); polyoxyethylene fatty acid glycerides and oils, such as polyoxyethylene (8) caprylic/capric mono- and diglycerides (e.g., Labrasol™, Gattefosse), polyoxyethylene (35) castor oil and polyoxyethylene (40) hydrogenated castor oil; polyoxyethylene alkyl ethers, such as polyoxyethylene (20) cetostearyl ether; polyoxyethylene fatty acid esters, such as polyoxyethylene (40) stearate; polyoxyethylene sorbitan esters, such as polysorbate 20 and polysorbate 80 (e.g., Tween™ 80, ICI); propylene glycol fatty acid esters, such as propylene glycol laureate (e.g., Lauroglycol™, Gattefosse); sodium lauryl sulfate; fatty acids and salts thereof, such as oleic acid, sodium oleate and triethanolamine oleate; glyceryl fatty acid esters, for example glyceryl monostearate; sorbitan esters, such as sorbitan monolaurate, sorbitan monooleate, sorbitan monopalmitate and sorbitan monostearate; tyloxapol; lecithin; stearyl triethanolamine; laurylaminopropionic acid; and mixtures thereof. Such surfactant component or wetting agent, if present, will typically together form about 0.25 weight percent to about 15 weight percent, preferably about 0.4 weight percent to about 10 weight percent, and more preferably about 0.5 weight percent to about 5 weight percent, of the total weight of the composition.

A thickening agent or viscosity-enhancing agent can be included to generally improve the mouth-feel of the composition and/or to help coat the lining of the gastrointestinal tract. While any suitable thickening agent can be included in the compositions of the present invention, a preferred thickening agent, when used, includes one or more of acacia, alginic acid bentonite, carbomer, carboxymethylcellulose calcium or sodium, cetostearyl alcohol, methyl cellulose, ethylcellulose, glycerin, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum. Such a thickening agent, if present, will typically form about 0.1 weight percent to 20 weight percent, preferably about 0.3 weight percent to about 15 weight percent, and more preferably about 0.5 weight percent to 4 weight percent, of the total weight of the composition.

A sweetening agent, such as one or more of sorbitol, saccharin, sucralose, sodium saccharin, xylitol, sucrose, aspartame, fructose, neotame, sodium saccharate, glycyrrhizin dipotassium, acesulfame K, mannitol, propylene glycol, invert sugar, and mixtures thereof, can be added to modify the taste of the composition. Alternatively or in addition, a viscous sweetener such as one or more of a sorbitol solution, syrup (sucrose solution) glycerin, or high-fructose corn syrup can be used and, in addition to sweetening effects, can also be useful to increase viscosity and to retard sedimentation. Preferably, the sweetening agent includes a saccharin, more preferably sodium saccharin. Such a sweetening agent, if present, will typically be present in an amount sufficient to mask the bitterness of the losartan, or salt or metabolite thereof, and preferably to also mask any other off-flavor components included in the formulation. Typical amounts of sweetening agent include about 0.05 weight percent to about 10 weight percent, preferably about 0.1 weight percent to about 5 weight percent, more preferably about 0.5 weight percent to 2 weight percent, of the total weight of the composition.

A flavoring agent can enhance patient compliance by making the composition more palatable, particularly in the case of the imbibable compositions of the present invention. The flavoring agent is typically selected in type and amount to decrease or eliminate any bitter taste, i.e., a taste mask, that would otherwise be detectable by the patient to whom the compositions are administered. Examples of a suitable flavoring agent, when used, includes one or more of aspartame, saccharin, menthol, peppermint, anise, and any fruit flavor, such as one or more of grapefruit, orange, lemon, lime, mango, strawberry, pineapple, or cherry; or a combination thereof. A flavoring agent including grapefruit has been found to work especially well according to the invention. The flavoring agent, if present, will typically form about 0.01 weight percent to about 15 weight percent, preferably about 0.5 weight percent to about 10 weight percent, and more preferably about 0.1 weight percent to about 5 weight percent, of the total weight of the composition.

A colorant agent, when included, can provide the compositions with a more aesthetic and/or distinctive appearance. Colorant agents suitable for inclusion in the present invention include one or more water-soluble synthetic organic food additives (e.g., food dyes such as food red dye Nos. 2 and 3, food yellow dye Nos. 4 and 5 and food blue dye Nos. 1 and 2), water-insoluble lake dyes (e.g., aluminum salts of the above water-soluble synthetic organic food additives, etc.), and natural pigments (e.g., beta-carotene, chlorophyll, iron oxide red, etc.). Suitable colorants include D&C Red No. 33, FD&C Red No. 3, FD&C Red No. 40, D&C Yellow No. 10, and C Yellow No. 6. Such a colorant agent, if present, will typically form about 0.001 weight percent to about 1 weight percent, preferably about 0.005 weight percent to about 0.5 weight percent, and more preferably about 0.0075 weight percent to about 0.25 weight percent, of the total weight of the composition.

Examples of a suitable preservative component, when used, includes sodium benzoate, butylated hydroxy toluene, butylated hydroxyanisole, ethylenediamine tetraacetic acid, paraoxybenzoic acid esters, chlorobutanol, benzyl alcohol, phenylethylalcohol, dehydroacetic acid, sorbic acid, benzalkonium chloride, benzethonium chloride, phenol, phenylmercuric nitrate, thimerosal, and mixtures thereof. A preservative can be added to the compositions at levels safe for ingestion to improve storage stability. A preservative component, if present, will typically form about 0.01 weight percent to about 5 weight percent, preferably about 0.05 weight percent to about 3 weight percent, and more preferably about 0.1 weight percent to about 2 weight percent, of the total weight of the composition.

The buffering agent preferably included in the liquid compositions can include a buffer solution of one or more gluconate, lactate, citrate, acetate, phosphate, and/or carbonate salts. In addition to providing the about 6 pH or greater, the buffering agent can also modulate drug solubility. Preferably, the buffering agent includes a phosphate, more preferably a potassium phosphate or sodium phosphate buffering agent. Such a buffering agent, if present, will typically form about 10 weight percent to about 80 weight percent, preferably about 30 weight percent to about 70 weight percent, and more preferably about 40 weight percent to about 60 weight percent, of the total weight of the composition.

Solubilizing and emulsifying agents are used to facilitate more uniform dispersion of an active ingredient or other excipient that is not generally soluble in the liquid carrier. Examples of a suitable emulsifying agent, if used, includes, for example, gelatin, egg yolk, casein, cholesterol, acacia, tragacanth, chondrus, pectin, methyl cellulose, carbomer, cetostearyl alcohol, cetyl alcohol, and mixtures thereof. Examples of a suitable solubilizing agent include glycol, glycerin, D-mannitol, trehalose, benzyl benzoate, ethanol, trisaminomethane, cholesterol, triethanolamine, sodium carbonate, sodium citrate, sodium salicylate, sodium acetate, and mixtures thereof. Preferably, the solubilizing agent includes a glycol. Preferred glycols include polyethylene glycol or propylene glycol, or both. The glycol can also act as a stabilizing agent. The solubilizing or emulsifying agent is/are generally present in an amount sufficient to dissolve or disperse the losartan, or salt or metabolite thereof, in the carrier. Typical amounts are from about 1 weight percent to about 80 weight percent, preferably about 20 weight percent to about 65 weight percent, and more preferably about 25 weight percent to about 55 weight percent, of the total weight of the composition.

A suitable isotonizing agent, if used, includes sodium chloride, glycerin, D-mannitol, D-sorbitol, glucose, and mixtures thereof. A suitable amount of the isotonizing agent, when included, is typically about 0.01 weight percent to about 5 weight percent, more preferably about 0.3 weight percent to about 4 weight percent, and more preferably about 0.5 weight percent to about 3 weight percent, of the total weight of the composition.

Examples of a suitable antioxidant component, if used, include one or more sulfites, tocopherol, vitamin E, and mixtures thereof. The antioxidant component provides long term stability to the liquid compositions. Addition of an antioxidant component can help enhance and ensure the stability of the compositions at ambient conditions for at least 18 to 24 months. A suitable amount of the antioxidant component, if present, is about 0.01 weight percent to about 3 weight percent, preferably about 0.05 weight percent to about 2 weight percent, and more preferably about 0.25 weight percent to about 1 weight percent, of the total weight of the composition.

Chelating agents can be added to trap metals that find their way into the compositions during processing. Suitable chelating agents include, but are not limited to, ethylenediaminetetraacetic acid (EDTA), ethylenediamine (EDA), diethylenetriamine (DETA), aminoethylethanolamine (AEEA), and mixtures thereof. The chelating agent can be present in an amount of about 0.01 weight percent to about 3 weight percent, preferably about 0.05 weight percent to about 2 weight percent, and more preferably about 0.25 weight percent to about 1 weight percent, of the total weight of the composition.

Through selection and combination of excipients according to the invention, liquid losartan compositions can be provided that exhibit improved or more desired performance with respect to drug concentration, dissolution, dispersion, stability, safety, emulsification, efficacy, flavor, patient compliance, and/or other pharmacokinetic, chemical and/or physical properties. It has now been further discovered that a carrier that includes one or more of a solubilizing agent, or a sweetening agent, along with a buffering agent, is particularly advantageous in an oral liquid losartan composition. Preferably, two or more of the solubilizing agent, sweetening agent, or buffering agent will be included. In one more preferred embodiment, the solubilizing agent, sweetening agent, and buffering agent are all included along with the losartan, or salt or metabolite thereof.

The glycol, when used, may be any pharmaceutically acceptable glycol such as ethylene glycol, propylene glycol, polyethylene glycol, polypropylene glycol, and mixtures thereof. In a preferred embodiment, the glycol includes propylene glycol. In one embodiment, the sweetening agent typically includes a saccharin-containing component, and the buffering agent typically includes a phosphate-containing buffer.

The phosphate-containing buffer typically includes sodium phosphate, potassium phosphate, or mixtures thereof. As used herein, “sodium phosphate” and “potassium phosphate” are meant to encompass any suitable phosphate salt including sodium dihydrogen phosphate monohydrate or sodium biphosphate, sodium monohydrogen phosphate heptahydrate or dibasic sodium phosphate, sodium monohydrogen phosphate dodecahydrate, potassium dihydrogen phosphate or potassium phosphate, and potassium monohydrogen phosphate, and any combination thereof. The concentration of phosphate salt in the buffering agent is typically from about 5 mM to 150 mM, preferably from about 50 mM to 120 mM, and more preferably is about 75 mM to 110 mM. An exemplary concentration of phosphate salt is 100 mM.

To provide a therapeutically or prophylactically effective amount of the active ingredient, losartan is generally present in an amount of about 1 to 50 mg/mL of the composition. The phrase “therapeutically effective amount” means that amount of losartan that provides a therapeutic benefit in the treatment or management of hypertension, congestive heart failure, diabetic nephropathy or myocardial infarction, and general malaise associated therewith. The term “prophylactically effective amount” means that amount of losartan that, alone or with another active ingredient, inhibits or prevents hypertension, congestive heart failure, diabetic nephropathy or myocardial infarction, and general malaise associated therewith.

The amounts of the other components in the composition may vary. Generally, the glycol can be present in an amount of about 1 percent to 40 percent (v/v) when included, the glycerin can be present in an amount of about 5 percent to 50 percent (v/v) when included, and/or the sweetening agent can be present in an amount of about 0.05 percent to 5 percent (w/v). The buffering agent is typically present in an amount of about 30 percent to 70 percent (v/v). As used herein, the terms (v/v) and (w/v) refer to percentages based on volume and percentages based on weight per volume, respectively. In one preferred embodiment, all four of these components are included in the liquid losartan compositions. In one embodiment, the losartan compositions of the invention—other than that present in any other carriers or the active ingredient—are at least substantially free of added water, preferably are entirely free of added water.

In a preferred embodiment, the losartan is present in an amount of about 8 mg/mL to 20 mg/mL of the composition, the glycol includes propylene glycol present in an amount of about 5 percent to 25 percent (v/v), the glycerin is present in an amount of about 20 percent to 40 percent (v/v), the sweetening agent includes saccharin sodium present in an amount of about 0.5 percent to 2 percent (w/v), and the buffering agent includes a phosphate-containing buffer present in an amount of about 40 percent to 60 percent (v/v). In an exemplary embodiment, the losartan is present in an amount of about 12.5 mg/mL, the propylene glycol is present in an amount of about 15 percent (v/v), the glycerin is present in an amount of about 30 percent (v/v), the potassium phosphate buffer is present in an amount of about 55 percent (v/v), and the saccharin sodium is present in an amount of about 1 percent (w/v).

The compositions of the present invention are preferably at least substantially stable. By “substantially stable” is meant a degradation of losartan in the composition over a period of at least two months that is no more than about 4 percent (measured on a weight basis) of the amount of losartan originally present in the composition, preferably no more than about 3 percent, and more preferably no more than about 2 percent, at 50° C. In an exemplary embodiment, the degradation of losartan over a period of at least two months is no more than about 1 percent to 2 percent of the amount of losartan originally present at 50° C. In each of these embodiments, it is preferred that the above-noted losartan degradation is over a period of at least three months.

The present invention also relates to methods of preparing oral liquid losartan compositions. Typically, this can include dissolving an amount of losartan, preferably in a therapeutically or prophylactically effective amount, into at least one liquid adjuvant to form a liquid losartan solution and combining the liquid losartan solution with one or more additional pharmaceutically acceptable carriers to form the oral liquid losartan composition having a pH of about 6 or greater.

The at least one liquid adjuvant may be selected from any of the agents already described above. In one embodiment, the at least one liquid adjuvant includes a buffering agent that can provide the desired pH to the compositions. In a preferred embodiment, the oral liquid losartan composition is a clear solution. By “clear” is meant that the light transmission through the composition is typically at least about 70 percent, preferably at least about 90 percent, and more preferably at least about 95 percent. In an exemplary embodiment, the composition is substantially transparent to the naked eye.

The present invention also provides methods of preventing, treating, or managing hypertension, congestive heart failure, diabetic nephropathy or myocardial infarction, in a mammal. As used herein, the terms “preventing, treating, or managing” cover preventing, treating, or managing the specified disease in a mammal, particularly a human, and includes: (i) preventing the disease from occurring in a subject that may be predisposed to the disease but has not yet been diagnosed as having it; (ii) inhibiting the disease, i.e., arresting its development before or after it afflicts a patient; or (iii) relieving the disease, i.e., causing regression of the disease. As used herein, “mammal” is meant the class of warm-blooded vertebrate animals that have, in the female, milk-secreting organs for feeding the young. Mammals include humans, apes, many four-legged animals, whales, dolphins, and bats. It should also be understood that symptoms of any disease are also encompassed within the term “managed,” such that managing hypertension may address some or all of the symptoms thereof with or without actually affecting the underlying disease itself.

The methods of the invention include administering to a mammal a therapeutically or prophylactically effective amount of an oral liquid losartan composition that includes losartan, or a pharmaceutically acceptable salt or metabolite thereof, and at least one pharmaceutically acceptable carrier. The oral liquid losartan composition has a pH of about 6 or higher. These methods find utility in preventing or treating numerous disease states that are currently being treated with losartan and include, for example, hypertension, congestive heart failure, diabetic nephropathy and myocardial infarction.

The prophylactically or therapeutically effective amount of losartan will vary depending on the subject being treated, the severity of the disease state and the manner of administration, and may be determined routinely by one of ordinary skill in the art. The dose, and perhaps dose frequency, will also vary according to the age, body weight, and response of the individual agent. In general, the total daily dose range is from about 25 mg to 100 mg administered in single or divided doses orally. The composition may typically be administered once or twice a day. It may be necessary to use dosages outside the above ranges in some cases, as will be apparent to those of ordinary skill in the art. Further, it is noted that the clinician or treating physician will know how and when to interrupt, adjust, or terminate therapy in conjunction with individual patient response.

The methods of the invention also contemplate the addition of one or more therapeutic agents with the losartan to provide an additive, more complete, or synergistic effect in preventing, treating, or managing a disease as noted herein. The additional “therapeutic agents,” which can be prophylactic or therapeutic, may be administered in any dosage form(s) suitable for the formulation as are well known in the art. Such dosage forms include solid dosage forms, such as tablets, capsules, powders, and cachets, or liquid dosage forms, such as suspensions, syrups, solutions, and elixirs. The agent may be incorporated in the losartan liquid composition or may be administered in a separate dosage form. The dosage form containing the additional agent to be administered will, in any event, contain a quantity of the additional therapeutic agent (s) in an amount effective to alleviate the symptoms of the subject being treated. The selection of these additional therapeutic agents will depend upon the specific disease state being treated, and are described in detail below. Preferably, all active ingredients will be in an oral liquid suspension form, more preferably in a combined form to facilitate patient compliance.

For those embodiments of the invention where the losartan liquid composition is administered to treat hypertension, such additional therapeutic agents can include one or more diuretics, beta-blockers, angiotensin converting (“ACE”) inhibitors, calcium channel blockers, alpha-blockers, alpha-beta blockers, vasodilators, alpha antagonists (centrally acting), or adrenergic neuron blockers, or a combination thereof; and preferably include one or more diuretics, beta-blockers, or calcium channel blockers, and combinations thereof.

For those embodiments of the invention where the losartan is administered to treat congestive heart failure, such additional therapeutic agents can include one or more of diuretics, ACE inhibitors, digoxin, vasodilators (direct vasodilators, calcium channel blockers and nitrates), beta blockers, or statins, or a combination thereof, and preferably diuretics, digoxin, direct vasodilators, or nitrates.

For those embodiments of the invention where the losartan is administered to treat diabetic nephropathy, such additional therapeutic agents can include one or more diuretics.

For those embodiments of the invention where the losartan is administered to treat myocardial infarction, such additional therapeutic agents can include one or more ACE inhibitors, diuretics, vasodilators, beta blockers, anticoagulants, or thrombolytics, or a combination thereof. A few examples of compounds within each of these classes is set forth below from which one of ordinary skill in the art may select as being suitable for administration in association with losartan, or a metabolite or salt thereof.

Examples of thiazide diuretics include bendroflumethiazide, chlorothiazide, chlorthalidone, hydrochlorothiazide, hydroflumethiazide, methyclothiazide, metolazone, polythiazide, quinethazone and trichlormethiazide; and are preferably selected from the group consisting of hydroclorothiazide and chlorothiazide. Commercially available thiazide diuretics include, for example, Naturetin™ (bendroflumethiazide, Squibb); Diuril™ (chlorothiazide, Merck); Thalitone™ (chlorthalidone, Boehringer); Microzide™; HydroDIURIL™; and Oretic™ (hydrochlorothiazide, Watson, Merck and Abbott, respectively); Saluron™ and Diucardin™ (hydroflumethiazide, Bristol-Myers and American Home Products, respectively); Enduron™ (methyclothiazide, Abbott); Mykrox™ and Zaroxolyn™ (metolazone, Fisons); Renese™ (polythiazide, Pfizer); Hydromox™ (quinethazone, American Cyanamid); and Naqua™ (trichlormethiazide, Schering); and combinations thereof.

Examples of loop diuretics include bumetanide, ethacrynic acid and furosemide. Commercially available loop diuretics include, for example, Bumex™ (bumetanide, Roche Pharmaceuticals), Edecrin™ (ethacrynic acid, Merck), Lasix™ (furosemide, Hoechst) and Myrosemide™ (furosemide); and combinations thereof.

Examples of potassium-sparing diuretics include amiloride, spironolactone and triamterene. Commercially available potassium-sparing diuretics include, for example, Midamor™ (amiloride, Merck); Aldactone™ (spironolactone, G. D. Searle) and Dyrenium™ (triamterene, Smith Kline); and combinations thereof.

Suitable beta-blockers may include propranolol, timolol, and metoprolol; and combinations thereof. Examples of suitable ACE inhibitors may include captopril, enalapril, lisinopril, quinapril, ramipril, benazepril and fosinopril; and combinations thereof. Suitable calcium channel blockers may include verapamil, diltiazem, nimodipine, nifedipine, nicardipine, felodipine, isradipine, and amlodipine; and combinations thereof. Exemplary alpha-blockers may include prazosin, terazosin, doxazosin, phenoxybenzamine and phentolamine; and combinations thereof. Suitable alpha-beta blockers include labetol.

Examples of suitable vasodilators may include, e.g., hydralazine, minoxidil, diazoxide and nitroprusside; and combinations thereof.

Suitable alpha antagonists (e.g., centrally acting) may include methyldopa, clonidine, guanabenz, and guanfacine; and combinations thereof.

Examples of suitable adrenergic neuron blockers may include guantacine, guanethidine, gunadrel, and reserpine; and combinations thereof.

In a preferred embodiment, the method includes administering at least one other antihypertensive agent with the composition. It is to be understood that the term “antihypertensive drug” refers to various classes of antihypertensive agents including angiotensin II antagonists, ACE inhibitors and NEP/ACE inhibitors which contribute, with the antiplatelet drug, to inhibit onset of primary or secondary cerebral infarction. The at least one other hypertensive agent preferably includes an angiotensin II antagonist, angiotensin converting enzyme inhibitor, or a neutral endopeptidase/angiotensin converting enzyme inhibitor.

A suitable angiotensin II receptor antagonist (also referred to herein as angiotensin II antagonist or All antagonist) suitable for association with a losartan composition according to the invention includes, but is not limited to, one or more of irbesartan, valsartan, telmisartan, candesartan, tasosartan or eprosartan, or a pharmaceutically acceptable salt thereof, with irbesartan being preferred.

The angiotensin-converting enzyme inhibitor, or ACE inhibitor, which may be employed herein includes those containing a mercapto (—S—) moiety such as substituted proline derivatives, for example, any of those disclosed in U.S. Pat. No. 4,046,889, with captopril, and mercaptoacyl derivatives of substituted prolines such as any of those disclosed in U.S. Pat. No. 4,316,906. Other examples of mercapto containing ACE inhibitors that may be included herein according to the invention include rentiapril (fentiapril, Santen) disclosed in Clin. Exp. Pharmacol. Physiol. 10: 131 (1983); as well as pivopril.

Other examples of suitable ACE inhibitors that may be associated with losartan include any of those disclosed in U.S. Pat. No. 4,374,829, any of the phosphonate substituted amino or imino acids or salts disclosed in U.S. Pat. No. 4,452,790, phosphinylalkanoyl prolines disclosed in U.S. Pat. No. 4,168,267, any of the phosphinylalkanoyl substituted prolines disclosed in U.S. Pat. No. 4,337,201, and the phosphonamidates disclosed in U.S. Pat. No. 4,432,971.

Other examples of suitable ACE inhibitors that may be employed herein include Beecham's BRL 36,378 as disclosed in European Patent Nos. 80822 and 60668; Chugai's MC-838 disclosed in CA. 102:72588v and Jap. J. Pharmacol., 40:373 (1986); Ciba-Geigy's CGS 14824 (3-([1-ethoxycarbonyl-3-phenyl-(1 S)-propyl]amino)-2,3,4,5-tetrahydro-2-oxo-1-(3S)-benzazepine-1 acetic acid HCl) disclosed in U.K. Patent No. 2103614 and CGS 16,617 (3(S)-[[(1S)-5-amino-1-carboxypentyl]amino]-2,3,4,5-tetrahydro-2-oxo-1H-1-benzazepine-1-ethanoic acid) disclosed in U.S. Pat. No. 4,473,575; cetapril (alacepril, Dainippon) disclosed in Eur. Therap. Res., 39:671 (1986); 40:543 (1986); ramipril (Hoechst) disclosed in European Patent No. 79-022 and Curr. Ther. Res., 40:74 (1986); Ru 44570 (Hoechst) disclosed in Arzneimittelforschung 35:1254 (1985), cilazapril (Hoffman-LaRoche) disclosed in J. Cardiovasc. Pharmacol. 9:39 (1987); Ro 31-2201 (Hoffman-LaRoche) disclosed in FEBS Lett. 165:201 (1984); lisinopril (Merck), indalapril (delapril) disclosed in U.S. Pat. No. 4,385,051; indolapril (Schering) disclosed in J. Cardiovasc. Pharmacol., 5:643, 655 (1983), spirapril (Schering) disclosed in Acta. Pharmacol. Toxicol., 59 (Supp. 5):173 (1986); perindopril (Servier) disclosed in Eur. J. Clin. Pharmacol., 31:519 (1987); quinapril (Warner-Lambert) disclosed in U.S. Pat. No. 4,344,949 and CI 925 (Warner-Lambert) ([3S-[2[R(*)R(*)]]3R(*)]-2-[2-[[1-(ethoxy-carbonyl)-3-phenylpropyl]amino[-1-oxopropyl]-1,2,3,4-tetrahydro-6,7-dimethoxy-3-isoquinolinecarboxylic acid HCl) disclosed in Pharmacologist 26:243, 266 (1984), WY-44221 (Wyeth) disclosed in J. Med. Chem., 26:394 (1983).

Preferred ACE inhibitors include fosinopril, captopril, enalapril, lisinopril, quinapril, benazapril, trandolapril, fentiapril, ramipril, moexipril; and combinations thereof.

Suitable NEP/ACE inhibitors may also be included herein in that they possess neutral endopeptidase (NEP) inhibitory activity and angiotensin converting enzyme (ACE) inhibitory activity. Examples of NEP/ACE inhibitors suitable for use herein include those disclosed in U.S. Pat. Nos. 5,508,272, 5,362,727, 5,366,973, 5,225,401, 4,722,810, 5,223,516, 5,552,397, 4,749,688, 5,504,080, 5,612,359, and 5,525,723 and European Patent Application Nos. 0481,522, 0534363A2, 534,396 and 534,492.

The term “about,” as used herein, should generally be understood to refer to both numbers in a range of numerals. Moreover, all numerical ranges herein should be understood to include each whole integer within the range.

Each of the patent applications, patents, publications, and other published documents mentioned or referred to in the Detailed Description is incorporated herein in its entirety by express reference thereto, to the same extent as if each individual patent application, patent, publication, and other published document was specifically and individually indicated to be incorporated by reference.

### EXAMPLES

The invention is further defined by reference to the following examples, describing in detail the methods used to prepare the compositions of the present invention. It will be apparent to those of ordinary skill in the art that many modifications, both to materials and methods, may be practiced without departing from the purpose and interest of the invention. These examples are for illustrative purposes only, and are not to be construed as limiting the appended claims.

### Example 1

### Solubility Studies for Losartan Potassium

### Example 1.1

### Comparative Losartan Solubility with and without a Buffering Agent

The solubility of losartan potassium was evaluated in water, sodium phosphate buffer, and ethanol. The materials used were losartan potassium; sodium phosphate buffer (SPB) prepared in-house at pHs of 1.9, 6.2, 7.2, and 8.2, at a concentration of 10 mM; and ethanol.

Small quantities of losartan potassium were added to 1 mL of the aqueous or alcohol media until crystals were visible by the eye. The results are provided in Table 1 below.

TABLE 1

| Solvent | pH | Solubility (mg/mL) |
|---|---|---|
| Water + HCl | 2.1 | 0.28 |
| Water | 5.5 | >243 |
| Water + NaOH | 11.8 | >239 |
| SPB | 8.2 | >253 |
| SPB | 7.2 | 300 |
| SPB | 6.2 | >241 |
| SPB | 1.9 | 0.048 |
| Ethanol | | 130 |

As can be seen from the results of Table 1, the solubility of losartan potassium surprisingly decreased significantly in acidic conditions (pHs of 1.9 and 2.1). In contrast, no major differences in solubility for losartan potassium were observed in alkaline conditions between phosphate buffer solutions and sodium hydroxide solution.

After solubility was determined, each sample was stored at 50° C. overnight. After 24 hours, the samples were examined. Losartan potassium crystals were observed in the SPB at a pH of 6.2. Thus, additional testing was performed on the compositions having a pH of at least 7.2 or higher to confirm that higher pH formulations can avoid significant amounts of losartan crystal precipitation. Without being bound by theory, it is believed that this surprising result is achieved when the pH is more than one pH unit greater than the pKa of losartan potassium, which is between 5 and 6. Formulations at these pHs of about 7 or greater should prevent precipitation of the drug. These results were obtained without the use of high performance liquid chromatography (HPLC) methods.

### Example 1.2

### Comparative Solubility of Different Buffering Agents

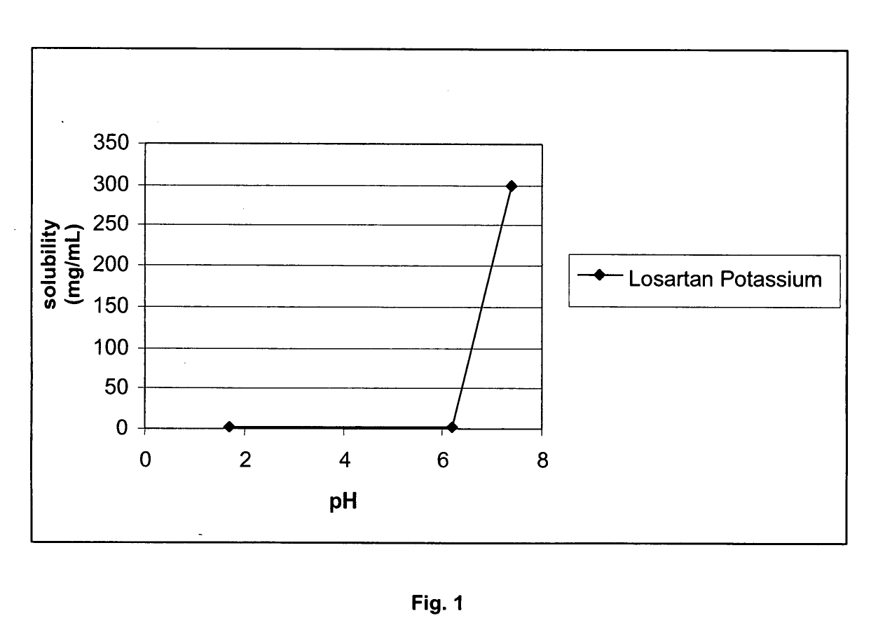

The solubility of losartan potassium in different phosphate-containing buffers was examined and the solubility of losartan was measured using HPLC. The materials used were sodium phosphate buffer (SPB) prepared in-house and losartan potassium. Forty (40) mg of losartan potassium was weighed in a glass tube, 10 mL of SPB was added, and the resulting mixture was vortexed for 2 minutes. A sample of each solution was then taken and analyzed by HPLC. If the losartan did not completely dissolve, the tubes were centrifuged and a sample of the supernatant was taken and analyzed by HPLC. Each sample was stored at room temperature and data was collected as a function of time. Table 2 below provides the results of the solubility of losartan potassium as a function at different pHs and different times. FIG. 1 illustrates the solubility profile of losartan potassium as a function of pH.

TABLE 2

| | SPB | SPB | SPB | SPB |
|---|---|---|---|---|
| | pH = 1.7 | pH = 6.2 | pH = 7.4 | pH = 8.2 |
| 0 hours | 1.07 | 1.62 | >250 | >250 |
| 48 hours | 0.152 | 1.65 | >250 | >250 |
| 480 hours | 0.03 | 0.06 | >250 | >250 |

The data collected by HPLC confirm the results of Example 1.1, which was performed visually. Based on the results of this and the previous example, it can be seen that losartan potassium is practically insoluble at a pH under 6.2. The solubility of losartan also decreased with time at a pH of 1.7 and 6.2. Moreover, some re-precipitation was observed at a pH of 6.2. At pHs of 7.4 and higher, no precipitation was observed.

Based on these studies, pHs of 7.4 or higher were determined to be the most advantageous to prepare losartan liquid solutions according to the invention.

### Example 2

### Stability Studies for Losartan Potassium

### Example 2.1

The effect of three variables on the stability of losartan potassium was investigated. The three variables were the pH of the buffer, cation of the buffer, and concentration of the phosphate salt in the buffer. The pHs studied were 7.4 and 8.2; the cations studied were potassium and sodium; and the concentrations studied were 10 mM and 100 mM.

The materials used were sodium phosphate buffer (SPB) and potassium phosphate buffer (PPB) prepared in-house and losartan potassium. Two hundred fifty (250) mg of losartan potassium was weighed in a glass tube, 10 mL of the appropriate buffer was added, and the resulting mixture was vortexed for 2 minutes. A sample of each solution was taken and analyzed by HPLC. The different solutions were then stored at 50° C. Samples were taken over time to study the stability of losartan potassium. The stability (measured as percent active on a weight

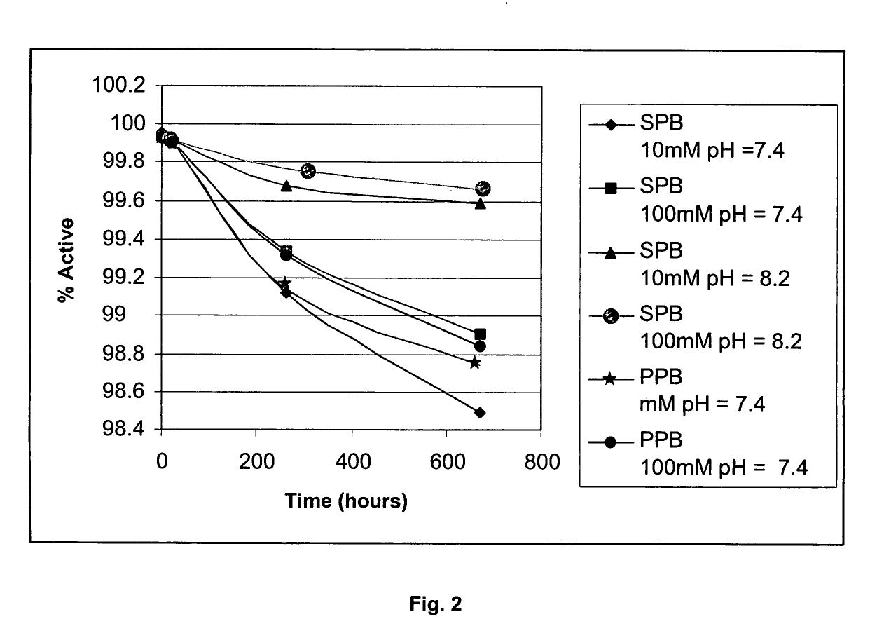

basis at different times) for each sample is provided in Table 3 below. FIG. 2 illustrates the stability of losartan as a function of time.

TABLE 3

| | 0 hours | 24 hours | 264 hours | 672 hours |
|---|---|---|---|---|
| SPB, 10 mM | 99.96 | 99.90 | 99.12 | 98.49 |
| pH = 7.4 | | | | |
| SPB, 100 mM | 99.93 | 99.90 | 99.34 | 98.90 |
| pH = 7.4 | | | | |
| SPB, 10 mM | 99.93 | 99.91 | 99.68 | 99.59 |
| pH = 8.2 | | | | |
| SPB, 100 mM | 99.93 | 99.91 | 99.77 | 99.66 |
| pH = 8.2 | | | | |
| PPB, 10 mM | 99.93 | 99.90 | 99.14 | 98.74 |
| pH = 7.4 | | | | |
| PPB, 100 mM | 99.93 | 99.90 | 99.32 | 98.84 |
| pH = 7.4 | | | | |

As can be seen from the above results, losartan potassium in SPB at a concentration of 10 mM and a pH of 7.4 showed the highest degradation with around 1.5 percent of the original losartan degraded after about 1 month at 50° C. When the concentration of the buffer was increased to 100 mM, the stability of losartan surprisingly increased so that the degradation was only around 1 percent after about 1 month at 50° C. Losartan potassium in PPB presented a similar degradation profile at a pH of 7.4 compared to SPB. The cations in this case, i.e., potassium and sodium, did not have a significant influence on the stability of losartan potassium. When losartan is used in the form of its potassium salt, it is preferred that PPB is used in the buffering agent according to the invention.

It can also be seen from the results in Table 3 that losartan potassium in SPB at a pH of 8.2 and a concentration of 100 mM provided a better stability than at a pH of 7.4. At a pH of 8.2, there was only about 0.4 percent degradation observed after about 1 month at 50° C. The higher concentration of 100 mM compared to 10 mM of SPB also increased the stability of losartan potassium in solution.

According to the results obtained, the stability of losartan improved at a pH of 8.2 and a concentration of the buffer salt of 100 mM.

### Example 2.2

The effect of different excipients at different concentrations on the stability of losartan potassium was studied.

A) Propylene Glycol (PG)

The effect of propylene glycol (PG) on the stability of losartan potassium in sodium phosphate buffer (SPB) at a pH of 7.2 was examined and studied. The materials used were SPB at a pH of 7.2 prepared in-house; losartan potassium; and PG. One hundred (100) mg of losartan potassium was weighed in a glass tube, X mL of SPB was added, the resulting mixture was vortexed for 2 minutes, (10-X) mL of PG was then added, and this mixture was vortexed for 1 minute. X represents the amount in mL of SPB added to provide 10 mL.

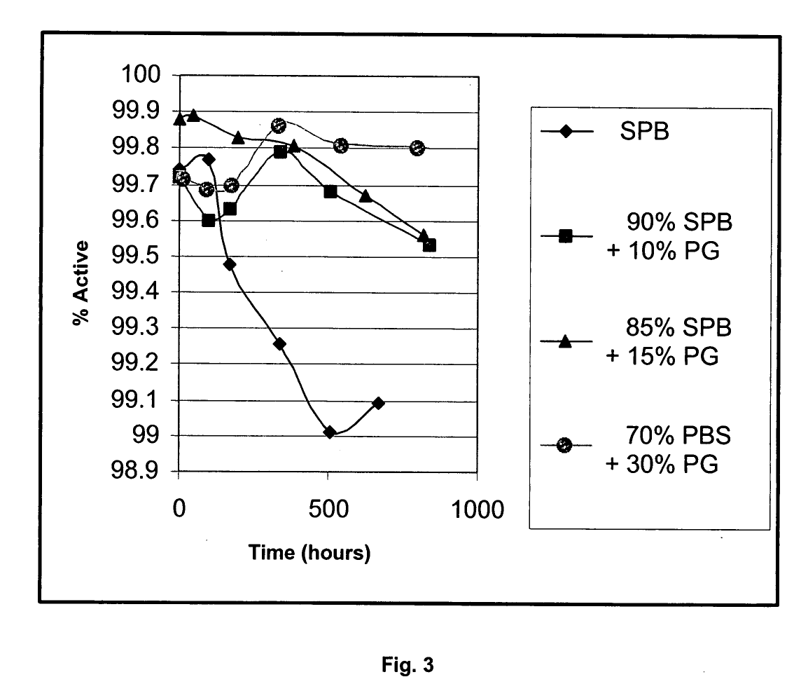

Three different compositions (10 percent, 15 percent, and 30 percent by volume PG) were prepared according to the method described above. Each formulation was evaluated by HPLC. The samples were then stored at 50° C. for about 1 month and analyzed periodically. The stability of losartan (measured by percent active) is illustrated graphically in FIG. 3 and is provided below in Table 4.

TABLE 4

| | 0 hours | 96 hours | 168 hours | 192 hours | 336 hours | 504 hours | 624 hours | 672 hours | 816 hours | 840 hours |
|---|---|---|---|---|---|---|---|---|---|---|
| Losartan | 99.74 | 99.77 | 99.48 | — | 99.26 | 99.01 | — | 99.09 | — | — |
| in SPB | | | | | | | | | | |
| Losartan | 99.72 | 99.60 | 99.63 | — | 99.79 | 99.68 | — | — | — | 99.53 |
| in 90% | | | | | | | | | | |
| SPB + | | | | | | | | | | |
| 10% PG | | | | | | | | | | |
| Losartan | 99.88 | — | — | 99.83 | — | — | 99.67 | — | 99.56 | — |
| in 85% | | | | | | | | | | |
| SPB + | | | | | | | | | | |
| 15% PG | | | | | | | | | | |
| Losartan | 99.72 | 99.69 | 99.69 | — | 99.87 | 99.82 | — | — | — | 99.80 |
| in SPB + | | | | | | | | | | |
| 30% PG | | | | | | | | | | |

The stability of losartan potassium in 30 percent PG was unexpectedly good. The stability was similarly surprisingly good at both 10 percent and 15 percent PG, with less than 0.4 percent degradation after more than 1 month (816 hours) at 50° C.

In all the samples shown above, introducing PG surprisingly and unexpectedly improved the stability of losartan potassium in solution. The maximum daily intake of PG recommended in 24 hours, however, is 25 mg/kg. For a 100 mg dose, the patient would have to take 4 mL of a solution at 12.5 mg/mL of losartan potassium. At 30 percent PG, the minimum weight of the patient would have to be approximately 50 kg. Therefore, a concentration of 15 percent PG is preferred to stay within the limits of the recommended maximum daily intake of PG.

B) Propylene Glycol (PG) and Glycerin, and Propylene Glycol (PG) and Polyethylene Glycol 400 (PEG 400)

The stability of losartan potassium in solution was studied in two combinations: (1) propylene glycol (PG) and glycerin; and (2) propylene glycol (PG) and polyethylene glycol 400 (PEG 400). The materials used were sodium phosphate buffer (SPB) at a pH of 7.2 prepared in-house; losartan potassium; PG; glycerin; and PEG 400. One hundred (100) mg of losartan potassium was weighed in a glass tube, X mL of SPB at a pH of 7.2 was added, the resulting mixture was vortexed for 2 minutes, 1.5 mL of PG was added, this mixture was vortexed for 1 minute, and finally (8.5-X) mL of either glycerin or PEG 400 was added. X represents the amount in mL of SPB added to provide 8.5 mL.

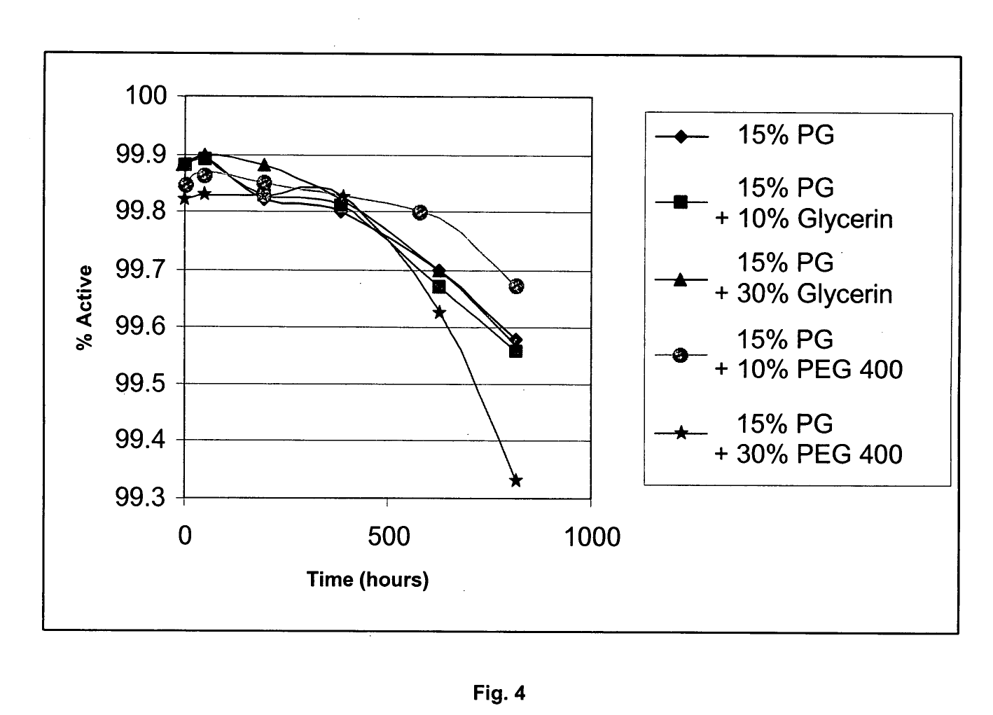

Two different concentrations of glycerin (10 percent and 30 percent by volume) and 2 different concentrations of PEG 400 (10 percent and 30 percent by volume) were studied. Five different formulations were prepared and analyzed. Each sample was evaluated by HPLC. The samples were then stored at 50° C. for about 1 month and analyzed periodically. The stability of losartan (measured by percent active) is illustrated graphically in FIG. 4 and is provided below in Table 5.

TABLE 5

| | 0 hours | 48 hours | 192 hours | 384 hours | 624 hours | 816 hours |
|---|---|---|---|---|---|---|
| Losartan in | 99.88 | 99.89 | 99.82 | 99.80 | 99.7 | 99.58 |
| SPB + 15% PG | | | | | | |
| Losartan in | 99.88 | 99.89 | 99.83 | 99.81 | 99.67 | 99.56 |
| SPB + 15% PG + | | | | | | |
| 10% Glycerin | | | | | | |
| Losartan in | 99.88 | 99.90 | 99.88 | 99.82 | 99.70 | 99.57 |
| SPB + 15% PG + | | | | | | |
| 30% Glycerin | | | | | | |
| Losartan in | 99.85 | 99.87 | 99.85 | 99.83 | 99.79 | 99.67 |
| SPB + 15% PG + | | | | | | |
| 10% PEG 400 | | | | | | |
| Losartan in | 99.82 | 99.83 | 99.83 | 99.83 | 99.63 | 99.33 |
| SPB + 15% PG + | | | | | | |
| 30% PEG 400 | | | | | | |

In SPB, losartan potassium in solution with 15 percent PG and up to 30 percent glycerin demonstrated less than 0.5 percent degradation after 816 hours at 50° C. The addition of glycerin at the studied concentrations did not significantly decrease the stability of losartan potassium. The addition of about 30 percent glycerin will also add sweetness to the composition and minimize or avoid the need to add a separate preservative.

Similar results were observed when glycerin was replaced by 10 percent PEG 400 with a slightly lower degradation (0.33 percent after 816 hours). Increasing the amount of PEG 400 to 30 percent in the composition, however, decreased the stability of losartan potassium. Thus, glycerin surprisingly demonstrated greater stability compared to PEG 400.

In addition, in another study (not described here) it was determined that both PG and PEG 400 improve the stability of losartan potassium in SPB. PEG 400, however, did not improve the stability of losartan potassium in water. Therefore, in one embodiment PG is preferred over PEG 400.

C) Propylene Glycol (PG), Glycerin and Xylitol

Even with 30 percent glycerin present, in some embodiments it is desired to add additional sweetener as the composition may not be sweet enough for some patients. Accordingly, a stronger sweetener was considered for use in the composition. Xylitol was considered to be a good candidate to increase the sweetness of the composition and also help stabilize losartan potassium in solution. Thus, the stability of a composition of propylene glycol (PG), glycerin, and xylitol was studied and evaluated. The materials used were sodium phosphate buffer (SPB) at a pH of 7.2 prepared in house; losartan potassium; PG; glycerin; and xylitol. One hundred (100) mg or 500 mg of losartan potassium were weighed in a glass tube, 200 mg or 400 mg of xylitol were added, 5.5 mL of SPB was added, and the resulting mixture was vortexed for 2 minutes. PG (1.5 mL) was then added, this mixture was vortexed for 1 minute, 3 mL of glycerin was added, and this mixture was finally vortexed for 1 minute.

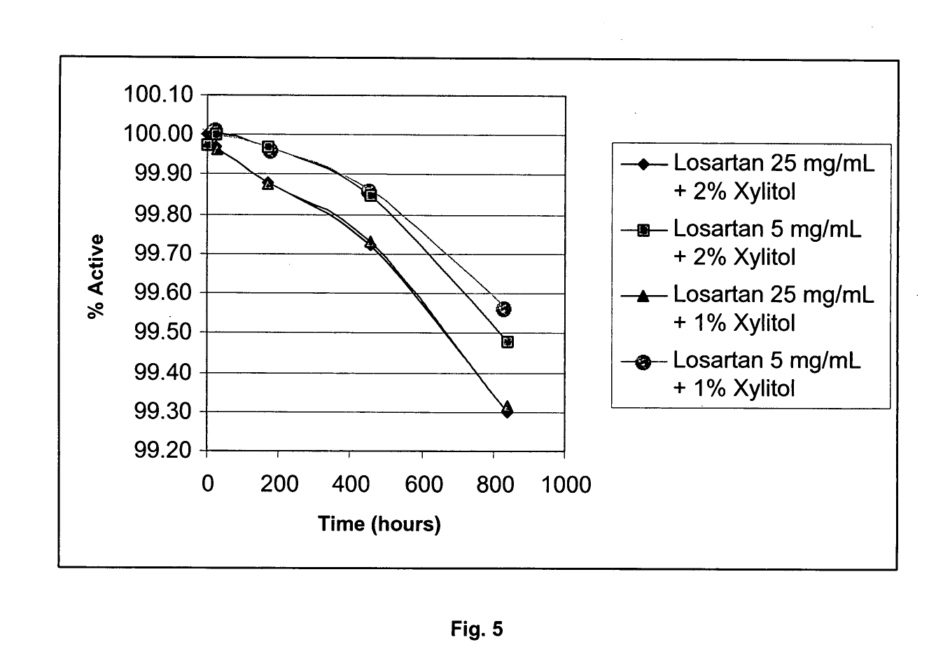

Two different concentrations of losartan (25 mg/mL and 5 mg/mL) and 2 different concentrations of xylitol (1 percent and 2 percent measured by weight per volume) were studied. Four different formulations were prepared and analyzed. Each sample was evaluated by HPLC. The samples were then stored at 50° C. for about 1 month and analyzed periodically. The stability of losartan (measured by percent active) is illustrated graphically in FIG. 5 and is provided below in Table 6.

TABLE 6

| | 0 hours | 24 hours | 168 hours | 456 hours | 840 hours |
|---|---|---|---|---|---|
| 25 mg/mL | 100.00 | 99.97 | 99.88 | 99.72 | 99.30 |
| losartan + | | | | | |
| 2% xylitol | | | | | |
| 5 mg/mL | 99.97 | 100.00 | 99.97 | 99.85 | 99.48 |
| losartan + | | | | | |
| 2% xylitol | | | | | |
| 25 mg/mL | 100.00 | 99.97 | 99.88 | 99.73 | 99.30 |
| losartan + | | | | | |
| 1% xylitol | | | | | |
| 5 mg/mL | 100.00 | 100.00 | 99.97 | 99.86 | 99.56 |
| losartan + | | | | | |
| 1% xylitol | | | | | |

Losartan potassium at a concentration of 25 mg/mL degraded approximately 0.7 percent after about 1 month at 50° C. in solution with either 1 percent xylitol or 2 percent xylitol. When the concentration of losartan was reduced to 5 mg/mL, the degradation decreased to about 0.5 percent in solutions containing 2 percent xylitol and about 0.45 percent for 1 percent xylitol.

According to these results, losartan potassium in combination with xylitol was more stable when a lower concentration of losartan was used. The concentration of xylitol, however, did not change the stability of losartan in the range of concentrations studied.

### Example 2.3

### Flavoring Agent According to the Invention

Based on the above solubility and stability experiments, phosphate buffers at a concentration of 100 mM of the salt and at a pH of 8.2 were found to have demonstrated surprisingly and unexpected improved solubility and stability compared to phosphate buffers at a pH of 7.4. It was also desired to minimize the bitterness of losartan potassium.

The composition containing 2 percent of xylitol was still not sweet enough. Saccharin sodium was considered to be a possible replacement for xylitol due to its extreme sweetness obtained during the excipient compatibility screening, which also surprisingly increased the stability of the formulation. The sweetness provided by glycerin could also be increased.

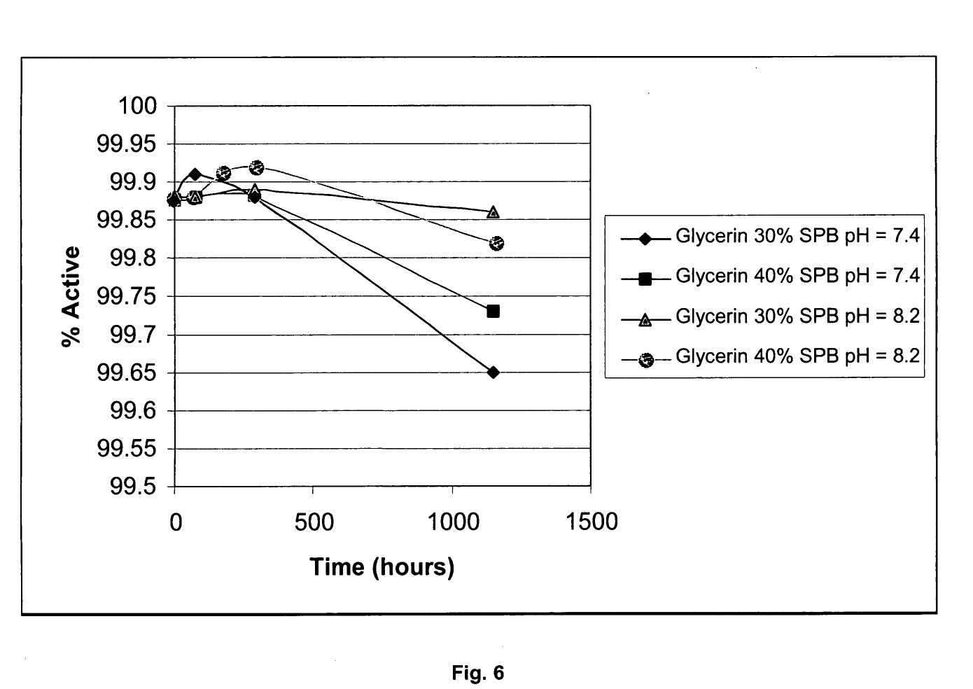

Two different concentrations of glycerin (30 percent and 40 percent by volume) and 2 different pHs of the phosphate buffer (7.4 and 8.2) were studied. Four different compositions that contained saccharin were prepared and analyzed. The materials used were losartan potassium; saccharin sodium; propylene glycol (PG); glycerin; and potassium phosphate buffer (PPB) prepared in-house. Two hundred fifty (250) mg of losartan potassium was weighed, 100 mg of saccharin sodium added, X mL of PPB was added, and the resulting mixture was vortexed for 2 minutes. Three mL of PG were then added to the vortexed mixture, (17-X) mL of glycerin added, and this mixture vortexed for 2 minutes. X represents the amount in mL of PPB added to provide 17 mL. Each sample was then stored at 50° C. and evaluated by HPLC periodically. The stability of losartan (measured by percent active) is illustrated graphically in FIG. 6 and is provided below in Table 7.

TABLE 7

| | 0 hour | 72 hours | 288 hours | 1152 hours |
|---|---|---|---|---|
| 30% glycerin, | 99.88 | 99.91 | 99.88 | 99.65 |
| pH = 7.4 | | | | |
| 40% glycerin, | 99.88 | 99.88 | 99.88 | 99.73 |
| pH = 7.4 | | | | |
| 30% glycerin, | 99.88 | 99.88 | 99.89 | 99.86 |
| pH = 8.2 | | | | |
| 40% glycerin, | 99.88 | 99.88 | 99.92 | 99.82 |
| pH = 8.2 | | | | |

Based on the results above, it was found that increasing the concentration of glycerin and replacing xylitol with saccharin surprisingly did not change the stability of losartan potassium in solution, however, the sweetness was beneficially increased to a more desired level. Xylitol can therefore be replaced by saccharin sodium without changing the stability of the composition. Minimal degradation was observed after 12 days (288 hours) and even at 1152 hours at 50° C.

Losartan potassium was again found to be more stable at a pH of 8.2, which confirmed the results of the previous experiments regarding pH and stability. It is possible to increase the concentration of glycerin without increasing the degradation of losartan potassium when using a buffer at a pH of 8.2.

According to all the studies performed, a preferred composition might include: propylene glycol (PG), glycerin, saccharin sodium, and potassium phosphate buffer at a pH of 8.2, along with sufficient active ingredient of losartan, or a salt or metabolite thereof.

### Example 3

### Oral Liquid Losartan Composition

An oral liquid losartan composition was prepared from losartan potassium at a concentration of 12.5 mg/mL, 15 percent (v/v) propylene glycol (PG), 30 percent (v/v) of glycerin, 55 percent (v/v) of potassium phosphate buffer (PPB) at a pH of 8.2, and 1 percent (w/v) saccharin sodium.

The materials used to prepare the composition were losartan potassium; PPB at a pH of 8.2 prepared in-house; PG; glycerin; and saccharin sodium. Two hundred (200) mg of PPB was added to a stainless steel beaker. To this was added 6.25 mg of losartan potassium while stirring at 300 rpm. The mixture was mixed for 5 minutes or until a clear solution was obtained. Five (5) grams of saccharin sodium was subsequently added, and the solution was mixed for 2 minutes or until it was clear. PG (77.7 g) was then added and the solution was mixed for 5 minutes. Next, glycerin (275.7 g) was added and the solution mixed for another 5 minutes. The container holding the glycerin and PG was then rinsed with 75.7 g of PPB, and this was added to the contents of the beaker. Finally, this solution was mixed for 5 minutes or until the composition was clear. The final volume of the composition was 500 mL.

Three samples of 25 mL each, was filled in 4 oz. glass bottles, 2 oz. plastic HDPE containers, and brown glass bottles. One sample of 25 mL was placed in a stainless steel container. One sample of each container (except for the stainless steel container) was then stored at 50° C., another one at 40° C. and finally the last sample at room temperature (RT). The stainless steel container was left at room temperature. Samples from each container were then analyzed by HPLC at different times. Table 8 displays the percentage of losartan potassium as a function of time in different containers. The margin of error in the data of the table below is ±0.5%.

TABLE 8

| | Temperature | 0 | 72 | 312 | 1008 |
|---|---|---|---|---|---|
| | (° C.) | hours | hours | hours | hours |
| Glass | RT | 99.82 | 99.92 | 99.80 | 99.70 |
| Bottle | | | | | |
| Glass | 50 | 99.82 | 99.95 | 99.80 | 99.90 |
| Bottle | | | | | |
| Glass | 40 | 99.82 | 99.92 | 99.92 | 99.90 |
| Bottle | | | | | |
| HDPE | RT | 99.82 | 99.92 | 99.78 | 99.90 |
| HDPE | 50 | 99.82 | 99.92 | 99.80 | 99.90 |
| HDPE | 40 | 99.82 | 99.92 | 99.83 | 99.90 |
| Brown | RT | 99.82 | 99.92 | 99.83 | 99.90 |
| Glass | | | | | |
| Bottle | | | | | |
| Brown | 50 | 99.82 | 99.92 | 99.83 | 99.90 |
| Glass | | | | | |
| Bottle | | | | | |
| Brown | 40 | 99.82 | 99.92 | 99.83 | 99.90 |
| Glass | | | | | |
| Bottle | | | | | |
| Stainless | RT | 99.82 | 99.92 | 99.83 | 99.90 |
| Steel | | | | | |

As demonstrated by the data above, the oral liquid losartan composition is stable as a function of time in all the different containers with minimal degradation observed after 1008 hours at room temperature, 40° C. and 50° C. These results demonstrate the superior stability of the losartan potassium in solution and confirm the conclusions of the other studies according to the invention.

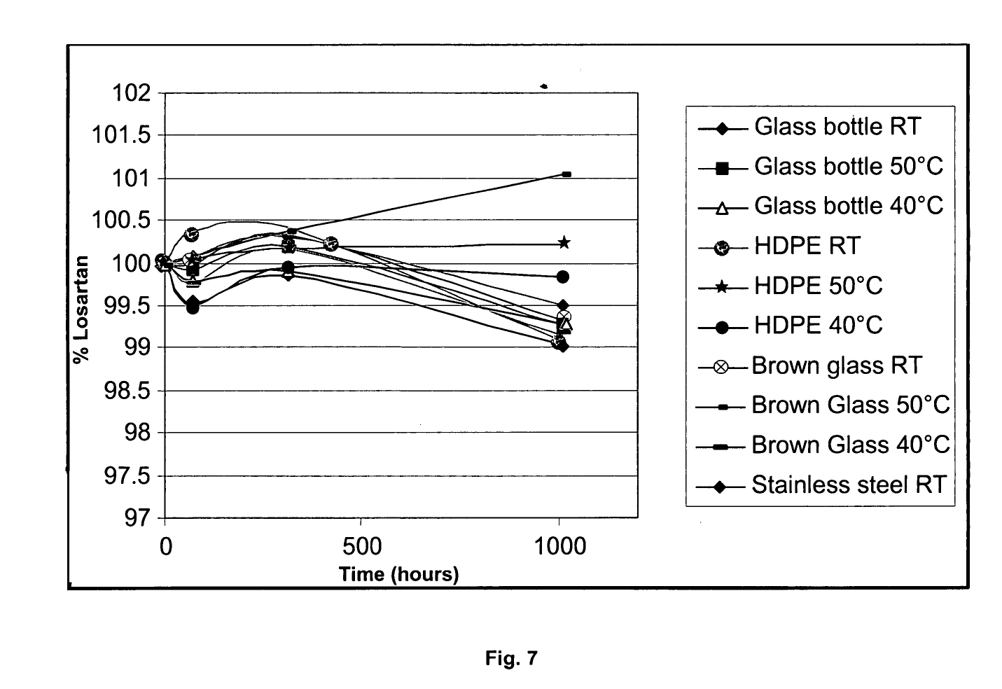

To further confirm these results, the percent recovery of losartan potassium was also measured as a function of time in the different containers. Table 9 and FIG. 7 summarize the results obtained. The margin of error in the data in this table is ±2%.

TABLE 9

| | Temperature | 0 | 72 | 312 | 1008 |
|---|---|---|---|---|---|
| | (° C.) | hour | hours | hours | hours |
| Glass | RT | 100.00 | 99.52 | 99.85 | 99.04 |
| Bottle | | | | | |
| Glass | 50 | 100.00 | 99.93 | 100.19 | 99.28 |
| Bottle | | | | | |
| Glass | 40 | 100.00 | 99.78 | 99.91 | 99.27 |
| Bottle | | | | | |
| HDPE | RT | 100.00 | 100.38 | 100.42 | 99.07 |
| HDPE | 50 | 100.00 | 100.05 | 100.16 | 100.22 |
| HDPE | 40 | 100.00 | 99.51 | 99.94 | 99.83 |
| Brown | RT | 100.00 | 100.00 | 100.32 | 99.33 |
| Glass | | | | | |
| Bottle | | | | | |
| Brown | 50 | 100.00 | 100.07 | 100.38 | 101.04 |
| Glass | | | | | |
| Bottle | | | | | |
| Brown | 40 | 100.00 | 99.77 | 100.17 | 99.15 |
| Glass | | | | | |
| Bottle | | | | | |
| Stainless | RT | 100.00 | 100.07 | 100.31 | 99.49 |
| Steel | | | | | |

Small variations in the percentage of recovery as a function of time were observed in the different containers. In each container at room temperature, there was a small decrease in the recovery percentage of losartan potassium. At less than 1 percent, the decrease, however, was very small. Accordingly, glass bottles, plastic HDPE containers, brown glass bottles, and stainless steel containers are all suitable containers for holding the oral liquid losartan compositions and do not significantly decrease their stability. The preferred form of packaging the liquid formulations of the invention is in PET, HDPE, clear or a brown glass bottle, preferably in a HDPE container.

Although preferred embodiments of the invention have been described in the foregoing description, it will be understood that the invention is not limited to the specific embodiments disclosed herein but is capable of numerous modifications by one of ordinary skill in the art. It will be understood that the materials used and the chemical or pharmaceutical details may be slightly different or modified from the descriptions herein without departing from the methods and compositions disclosed and taught by the present invention.

What is claimed is:

1. An oral liquid composition comprising losartan, or a pharmaceutically acceptable salt or metabolite thereof, and at least one pharmaceutically acceptable carrier in an amount sufficient to provide a pH of about 6 or higher.

2. The composition of claim 1, wherein the pH is about 7 to 10.

3. The composition of claim 2, wherein the pH is about 7.4 to 8.6.

4. The composition of claim 1, wherein the at least one pharmaceutically acceptable carrier comprises one or more of a wetting agent, surfactant component, stabilizing agent, solubilizing agent, thickening agent, sweetening agent, flavoring agent, perfuming agent, colorant agent, preservative agent, or buffering agent.

5. The composition of claim 4, wherein the carrier comprises one or more of a solubilizing agent, a stabilizing agent, a sweetening agent, a flavoring agent effective to mask a bitter taste, or a buffering agent.

6. The composition of claim 5, wherein the carrier comprises a glycol or glycerin, the sweetening agent comprises a sucralose or saccharin-containing component, and the buffering agent comprises a phosphate-containing buffer.

7. The composition of claim 5, wherein the flavoring agent is present and comprises grapefruit, orange, lemon, lime, mango, strawberry, pineapple, cherry, or a combination thereof.

8. The composition of claim 6, wherein the phosphate-containing buffer comprises sodium phosphate, potassium phosphate, or mixtures thereof.

9. The composition of claim 8, wherein the concentration of phosphate salt in the buffering agent is from about 5 mM to 150 mM.

10. The composition of claim 9, wherein the phosphate salt concentration is about 80 mM to 120 mM.

11. The composition of claim 5 wherein the solubilizing agent is present in an amount of about 1 percent to 50 percent (v/v).

12. The composition of claim 5, wherein the sweetening agent is present in an amount of about 0.05 percent to 5 percent (w/v).

13. The composition of claim 5, wherein the buffering agent is present in an amount of about 30 percent to 70 percent (v/v).

14. The composition of claim 6, wherein the glycol is present in amount of about 1 percent to 40 percent (v/v), and the glycerin is present in an amount of about 5 percent to 50 percent (v/v).

15. The composition of claim 1, wherein the losartan, or salt or metabolite thereof, is present in an amount of about 1 to 50 mg/mL of the composition.

16. The composition of claim 1, wherein the losartan, or salt or metabolite thereof, is present in an amount of about 8 mg/mL to 20 mg/mL of the composition, and the pharmaceutically acceptable carrier comprises a glycol present in an amount of about 5 percent to 25 percent (v/v), glycerin present in an amount of about 20 percent to 40 percent (v/v), a sweetening agent present in an amount of about 0.5 percent to 2 percent (w/v), and a buffering agent present in an amount of about 40 percent to 60 percent (v/v).

17. The composition of claim 1, wherein the composition is at least substantially stable.

18. The composition of claim 1, wherein degradation of losartan over a period of less than two months is no more than about 1 percent to 2 percent (w/w) at 50° C.

19. The composition of claim 1, which is in solution form.

20. An oral liquid composition comprising losartan, or a pharmaceutically acceptable salt or metabolite thereof, and at least one of propylene glycol or glycerin, along with a sucralose or saccharin-containing component and a phosphate-containing buffer comprising sodium phosphate, potassium phosphate, or a mixture thereof, in an amount sufficient to provide a pH of about 6 or higher.

21. A stable oral liquid losartan composition comprising:
losartan or a pharmaceutically acceptable salt or metabolite thereof;
a solubilizing agent;
a sweetening agent; and
a buffering agent, present in an amount sufficient to provide a pH of about 6 or higher over an extended period of time to minimize degradation of the losartan.

22. A oral liquid composition comprising a solution of losartan, or a pharmaceutically acceptable salt or metabolite thereof, propylene glycol, glycerin, saccharin sodium, and potassium phosphate buffer, each in an amount sufficient to provide a pH of about 6 or higher.

23. A method of preparing an oral liquid losartan composition which comprises:
dissolving an amount of losartan, or a pharmaceutically acceptable salt or metabolite thereof, into at least one liquid adjuvant to form a liquid losartan solution; and
combining the liquid losartan solution with one or more additional pharmaceutically acceptable carriers sufficient to provide a pH of about 6 or higher to form the oral liquid losartan composition.

24. The method of claim 23, wherein the at least one liquid adjuvant comprises a buffering agent.

25. The method of claim 23, wherein the oral liquid losartan composition is a clear solution.

26. A method of preventing, treating, or managing hypertension, congestive heart failure, diabetic nephropathy or myocardial infarction in a mammal which comprises administering to the mammal an effective amount of an oral liquid losartan composition comprising losartan, or a pharmaceutically acceptable salt or metabolite thereof, and at least one pharmaceutically acceptable carrier, wherein the oral liquid losartan composition has a pH of about 6 or higher.

27. The method of claim 26, wherein the composition is administered once or twice a day.

28. The method of claim 26, wherein the total daily dose of losartan is between about 25 mg to 100 mg.

29. The method of claim 26, which further comprises administering at least one other therapeutic agent.

30. The method of claim 29, wherein the at least one other therapeutic agent is at least one other hypertensive agent.

31. The method of claim 30, wherein the at least one other hypertensive agent comprises an angiotensin II antagonist, angiotensin converting enzyme inhibitor, or a neutral endopeptidase/angiotensin converting enzyme inhibitor.
